(19) United States
(12) Patent Application Publication
(10) Pub. No.: US 2015/0004631 A1
(43) Pub. Date: Jan. 1, 2015

(54) Plasmodium falciparum HLA class I restricted T-cell epitopes

(71) Applicants: Martha Sedegah, Gaithersburg, MD (US); Thomas Richie, Glenelg, MD (US)

(72) Inventors: Martha Sedegah, Gaithersburg, MD (US); Thomas Richie, Glenelg, MD (US)

(21) Appl. No.: 13/930,468

(22) Filed: Jun. 28, 2013

(51) Int. Cl. G01N 33/50

(52) U.S. Cl. CPC G01N 33/505
USPC 435/7.92

(57) ABSTRACT

The invention relates immunogenic polypeptides and epitopes from Plasmodium falciparum protein AMA 1. The epitopes contain HLA class I binding motifs and stimulate an anti-malaria CD8⁺ T-cell response. The polypeptides can be incorporated into immunogenic formulations against malaria. Additionally, the antigens are useful for facilitating evaluation of immunogenicity of candidate malaria vaccines.

### CROSS-REFERENCES TO RELATED APPLICATIONS

The application is a Divisional application of U.S. application Ser. No. 12/942,103, filed Nov. 9, 2010, which claims priority to U.S. provisional application No. 61/281,163 filed Nov. 13, 2009, which are incorporated by reference, herein.

### BACKGROUND OF INVENTION

1. Field of Invention

The inventive subject matter relates to Plasmodium falciparum AMA1 polypeptides containing HLA-restricted CD8⁺ T-cell epitopes. The inventive polypeptides or epitopes can be utilized in assays to evaluate candidate vaccines to malaria. Additionally, the polypeptides can be incorporated into vaccine formulations against P. falciparum.

2. Background Art

Malaria is caused by the vector borne organism Plasmodium spp. The parasite has a complex lifecycle involving stage specific expression of proteins. These proteins can be expressed at different stages or be specific to stages. Malaria is an extremely important disease, with over 3 billion people living in malaria endemic areas. Over 1 million deaths are attributable to malaria per year. The emergence of drug resistant strains has compounded the problem of treating the disease. Unfortunately, no FDA-approved vaccine exists.

The entire genomic sequence of P. falciparum has been sequenced (Bowman et al., Nature, 400: 532-538 (1999), Gardner, et al., Nature, 419: 498-511 (2002)). The rodent malaria parasite, P. yoelii has also been sequenced (Carlton et al., Nature, 419: 512-519 (2002)). Despite this, however, the development of efficacious anti-malaria vaccines has been severely hampered by the paucity of promising antigens. As such, no FDA-approved vaccine to this agent exists.

Sterile protective immunity to malaria induced by experimental immunization with irradiated sporozoites is thought to be mediated by CD4+ and CD8+ T cells directed against malaria antigens expressed on the surface of infected hepatocytes and perhaps anti-sporozoite antibodies [1]. Naturally acquired anti-malarial immunity is mediated primarily by antibodies to blood-stage parasites with T cell responses possibly providing a contribution. Both CD4+ and CD8+ T cells are needed for optimal effector cell functions. Furthermore, the development of immunological memory (Beeson, et al., Trends Parasitol 24: 578-584 (2008) and T cell responses is known to be genetically restricted.

### SUMMARY OF THE INVENTION

The invention relates to polypeptides containing HLA-restricted CD8+ T-cell epitopes from the P. falciparum protein AMA. In one embodiment, one or more polypeptides can be included in immunogenic composition against malaria. In this embodiment, one or more proteins can be produced by first inserting the DNA encoding the proteins in suitable expression systems. The expressed and purified proteins can then be administered in one or multiple doses to a mammal, such as humans. In this embodiment, the purified proteins can be expressed individually or DNA encoding specific proteins can be recombinantly associated to form a single immunogenic composition. These immunogenic compositions can then be administered in one or multiple doses to induce an immunogenic response.

In an alternative embodiment, DNA encoding the proteins can be inserted into suitable vector expression systems. These include, for example, adenoviral based systems, such as in Bruder, et al (patent application publication number US 20080248060, published Oct. 9, 2008) or a DNA plasmid system.

In order to develop anti-malaria vaccines that stimulate an enhanced cell mediated response, it is important for the vaccine to contain appropriate CD4⁺ and CD8⁺ T-cell epitopes. CD8+ T cell responses that produce IFN-γ and multifunctional responses (i.e., produce more than 2 cytokines) have been associated with protection in other diseases (Darrah, et al., Nat. Med., 13: 843-50 (2007); Seder, et al., Nat Rev Immunol., 8: 247-58 (2008); Lindenstrom, et al., J. Immunol., 182: 8047-55 (2009); Valor, et al., Vaccine, 26: 2738-45 (2008); Bansal, et al., J. Virol., 82: 6458-69 (2008); Walther, et al., Infect. Immun., 74: 2706-16 (2006); Karanam, et al., Vaccine 27: 1040-9 (2009)). For this reason, it is important develop immunogenic candidates that contain T-cell epitopes.

In a preferred embodiment, polypeptides of specific regions of the P. falciparum AMA1 protein were identified and isolated, which contain CD8⁺ T cell epitopes. Because of the importance of CD8⁺ T-cells in conferring immunity to malaria, these polypeptides are useful as components of immunogenic compositions against malaria.

There are many hundreds of HLA A and B alleles that can be classified into 12 Class I super-types that cover most of the known HLA-A and HLA-B polymorphisms, permitting identification of potential peptide binding motifs that should recognize the super-types (Sette, A. and J. Sidney, Immunogenetics 50: 201-12 (1999)). Algorithms have been developed to aid prediction of peptide sequences that bind to CD4⁺ or CD8⁺ T cells (Gowthaman, U. and J. N. Agrewala, Expert Rev Proteomics 6: 527-37 (2009); Tian, et al., Amino Acids 36: 535-54 (2009); Hattotuwagama, et al., Methods Mol. Biol. 409: 227-45 (2007)).

The computerized algorithm NetMHC (Hoof, et al., Immunogenetics 61: 1-13 (2009)) was used to predict the binding affinities of 8-10mer peptides contained within 15 mer peptides grouped into AMA peptide pools. These peptides were then evaluated for their ability to stimulate CD8⁺ T-cells. The peptides contain not only anti-malaria T-cell epitopes but also specific HLA class I protein binding motifs. Therefore, these polypeptides are valuable as components in immunogenic formulations capable of eliciting a response to defined populations. An embodiment of the current invention is to utilize one or more of the identified polypeptides in immunogenic formulations in order to develop immunogenic responses to as broad a population as possible.

### BRIEF DESCRIPTION OF DRAWINGS

FIG. 1. CD4⁺ and CD8⁺ T cell IFN-γ and multifunctional responses. AMA1 peptide pool (P) and its minimal CD8+ T epitope (E) for each volunteer were tested in intracellular cytokine staining assay (ICS). Activity was measured as a percentage of cytokine producing CD4⁺ and CD8⁺ T cells producing IFN-γ, or multifunctional responses defined as any two cytokines from IFN-γ, IL-2 and TNF-α.

FIG. 2. AMA14e is as active in ELISpot as peptide pools and minimal epitopes. The minimal epitopes include “NEVVVKEEY” (SEQ ID No. 6) and “HPKEYEYPL” (SEQ ID No. 14). PBMC from each volunteer were tested in ELISpot with API-12 (SEQ ID No. 22) (mixture of 153 15mer over lapping peptides, AMA14e (mixture of 14 minimal epitopes), Ap1 or Ap10, and individual epitope (consistent with that volunteer's HLA). AMA14e was as active as Ap1-12 with v002 and v005 and nearly as active with v001. Bars represent standard deviation of the mean response.

FIG. 3. ELISpot activity against AMA14e after CD4⁺ or CD8⁺ T cell depletion. PBMC from v001 or v005 were tested in ELISpot after CD4⁺ or CD8⁺ T cell depletion, and in ICS for total CD4⁺ or CD8⁺ T cell IFN-γ, with AMA14e, Ap1-12 (all 153 15mer peptides), AMA1 recombinant protein (rec. protein). Depletion and ICS assays show that AMA14e and Ap1-12 are both strongly recognized by CD8⁺ T cells. However, recombinant AMA1 protein is preferentially recognized by CD4⁺ T cells.

FIG. 4. Distribution of HLA-A and HLA-B epitopes in AMA1 identified in this study.

FIG. 5. Illustration of regions of AMA-1 encompassing peptide pools Ap1-12 (SEQ ID No. 22). The top bar illustrates the AMA1 construct expressed by the adenovirus expression system. The second bar illustrates the AMA1 construct expressed by the DNA expression system.

FIG. 6. Illustration of Plasmodium falciparum AMA1 protein regions represented by peptide pools Ap1-12. Also illustrated in the response (in spot forming cells/million) of PBMC's from selected individual volunteers.

FIG. 7. AMA1 pools that induced a high response by PBMC in ELISpot assay. The star over certain bars denotes individuals that, upon challenge with P. falciparum, exhibited protective immunity.

FIG. 8. AMA1 pools that induced a relatively lower response by PBMC in ELISpot assay, as compared to those represented in FIG. 7.

### DETAILED DESCRIPTION OF PREFERRED EMBODIMENTS

The following terms are defined: Antigen is a chemical moiety containing at least one epitope capable of stimulating or reacting with immune products, such as antibody or T-cells; T-cell Epitope is defined in this application as a minimal polypeptide region capable of stimulating a T-cell response. As used in this application, an epitope is typically 8 to 10 amino acids; AMA1 refers to apical membrane antigen 1, which is a protein expressed by Plasmodium falciparum; AMA14e is a composition, containing a mixture of 14 AMA 1 HLA-restricted CD8⁺ T-cell epitopes that is capable of inducing a cell medicated immune response; An HLA motif is an amino acid sequence associated with binding to HLA molecules and can be associated with T-cell recognition of antigen in an HLA-restricted fashion; An Immunogenic composition refers to a chemical, compound or formulation that, once administered, will elicit an immune response; A vaccine is an immunogenic composition used to induce protective immunity; A DNA expression system is a molecular system containing plasmid or closed loop DNA containing elements for expressing an inserted DNA sequence as polypeptide; HLA refers to human leukocyte antigens; A viral expression system is any viral based system, including viral-like particles or viral replicons, containing elements for expressing an inserted DNA sequence as a polypeptide.

CD4⁺ and CD8⁺, HLA-restricted T cells are needed for optimal effector cell functions. Therefore it is necessary to ensure that malaria vaccines contain appropriate HLA-restricted CD4+ and CD8+ T cell epitopes that are recognized by as wide a population as possible. Secondly, identification of such epitopes that can be combined into a single pool for stimulating PBMC has the potential to facilitate the determination of immunogenicity of candidate malaria vaccines where cell numbers can are limited.

In order to develop anti-malaria vaccines that stimulate an enhanced cell mediated response, it is important to map the HLA-restricted T-cell epitopes of potentially important malarial antigens, such as AMA1. CD8⁺ T cell responses that produce IFN-γ and multifunctional responses (i.e., produce more than 2 cytokines) have been associated with protection in other diseases (Darrah, et al., Nat. Med., 13: 843-50 (2007); Seder, et al., Nat Rev Immunol., 8: 247-58 (2008); Lindenstrom, et al., J. Immunol., 182: 8047-55 (2009); Valor, et al., Vaccine, 26: 2738-45 (2008); Bansal, et al., J. Virol., 82: 6458-69 (2008); Walther, et al., Infect. Immun., 74: 2706-16 (2006); Karanam, et al., Vaccine 27: 1040-9 (2009)). For this reason, it is important development of immunogenic candidates containing T-cell epitopes, especially, CD8⁺ epitopes.

In a preferred embodiment, polypeptides of the Plasmodium falciparum protein, AMA-1 (PfAMA1), regions wre identified and isolated that contain CD8⁺ T-cell epitopes. One or more of these polypeptides from these regions can be used in immunogenic compositions against malaria.

Hundreds of HLA A and B alleles exist and that can be classified into 12 Class I super-types that cover most of the known HLA-A and HLA-B polymorphisms, allowing identification of a potential peptide binding motifs that should recognize the super-types (Sette, A. and J. Sidney, Immunogenetics 50: 201-12 (1999)). Algorithms have been developed to aid prediction of peptide sequences that bind to CD4+ or CD8+ T cells (Gowthaman, U. and J. N. Agrewala, Expert Rev Proteomics 6: 527-37 (2009); Tian, et al., Amino Acids 36: 535-54 (2009); Hattotuwagama, et al., Methods Mol. Biol. 409: 227-45 (2007)).

The computerized algorithm NetMHC (24) was used to predict the binding affinities of 8-10mer peptides contained within 15 mer peptides grouped into AMA peptide pools.

For example, the characterized epitopes were restricted by 4 of the 7 HLA super-types expressed by the volunteers that included A1, A2, B8 and B44. Since these super-types have different distribution frequencies according to different populations, additional analysis of peptides within these pools, or even pools from other antigens, may identify HLA class I-restrictions in super-families that represent a larger spectrum of the human population.

### Example 1

### Identification of AMA1 Peptide Pools

Six volunteers (designated as Gp1) were immunized with low dose (2×10¹⁰ particle units) of Ad5-PfCA vaccine. The vaccine construct contained CSP and AMA1 antigens. Five of the six volunteers were utilized in the study. The sixth volunteer gave poor responses at all time points following immunization and was excluded.

The five volunteers expressed alleles representing a total of 7 super-types (Sidney, et al., Immunol. 9: 1 (2008)). The super-types represented were A01, A02, A03, B08, B27, B44 and B58. These represent 3/6 of the A and 4/6 of the B described super-types. Table 1 summarizes the HLA-A and B alleles and super-types of this group. Table 1, shows identification of the HLA super-type for each volunteer.

TABLE 1
Volunteers HLA A and B alleles and super-types

| Volunteer | A Super-type | B Super-type |
|---|---|---|
| 001 | A01/A02 | B44/B44 |
| 002 | A01/A02 | B08/B44 |
| 005 | A01/A02 | B08/B27 |
| 008 | A02/A03 | B27/B27 |
| 012 | A01/A03 | B44/B58 |

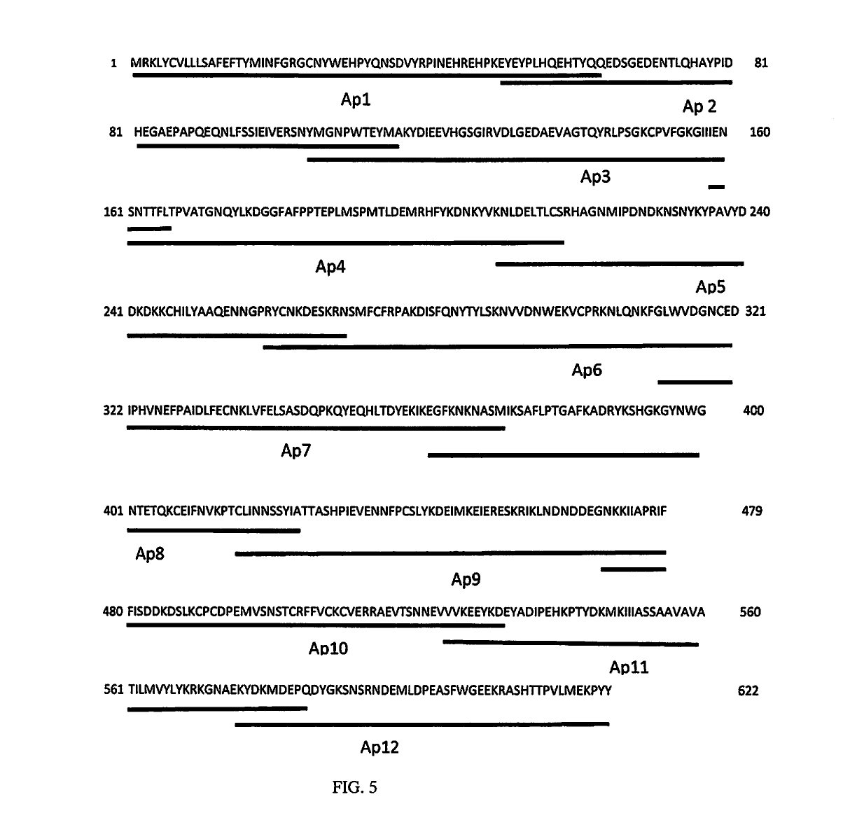

A total of 153 15-mer overlapping peptides spanning the entire full-length PfAMA1 were synthesized. The peptides were then combined into 12 peptide pools for initial assays. The regions of AMA-1 encompassing the 12 pools are illustrated in FIG. 5.

In order to assess the pools for their ability to induce T-cell responses, specifically CD 8⁺ responses, PBMC, 1 month post immunization were used, from the five volunteers in Table 1, in ELISpot assays. In these studies, IFN-γ activity was measured in response to exposure to the peptide pools. The assays were conducted essentially as described by Wang, et al., Proc. Natl. Acad. Sci. (U.S.A.), 98: 10817-22.

The results of the study are summarized in Table 2a. In Table 2a, the PBMCs most strongly reacted with 7 of the 12 AMA peptide pools (i.e., Ap1, Ap-3, Ap-4, Ap-7, Ap 8, Ap-10 and Ap 11 (Table 2a). The 91 15-mers contained within these pools were then tested individually, and a total of 23 were recognized by one or more of the 5 volunteers (using criteria for positive as 40 spot forming cells/million PBMC). The results of this study are shown in Table 2b.

TABLE 2a
ELISpot IFN-γ activity of AMA1 peptide pools (SFC/10⁶)

| Volunteer | Ap1 | Ap2 | Ap3 | Ap4 | Ap5 | Ap6 | Ap7 | Ap8 | Ap9 | Ap10 | Ap11 | Ap12 |
|---|---|---|---|---|---|---|---|---|---|---|---|---|
| 1 | 99 | 104 | 87 | 134 | 166 | 106 | 163 | 109 | 103 | 271 | 153 | 61 |
| 2 | 139 | 127 | 99 | 198 | 20 | 40 | 110 | 6 | 29 | 23 | 33 | 6 |
| 5 | 564 | 64 | 159 | 498 | 114 | 143 | 134 | 286 | 59 | 53 | 101 | 18 |
| 8 | 14 | — | 137 | 43 | 18 | 41 | 39 | 149 | 38 | 73 | 7 | 20 |
| 12 | 2 | 13 | 6 | 13 | 7 | 11 | 31 | 286 | 30 | 227 | 83 | 2 |

TABLE 2b

| Volunteer | Pool | SFC/10⁶ | Pool-pep # | Sequence | 15 mer | SFC/10^{⁶} | Pep # |
|---|---|---|---|---|---|---|---|
| 001 | Ap10 | 193 | Ap10-13 | 517-531 | TSNNEVVVKEEYKDE | 250 | 1 |
| | Ap7 | 131 | Ap7-3 | 321-335 | EDIPHVNEFPAIDLF | 139 | 2 |
| | Ap7 | 131 | Ap7-4 | 325-339 | HVNEFPAIDLFECNK | 96 | 3 |
| | Ap7 | 131 | Ap7-7 | 337-351 | CNKLVFELSASDQPK | 77 | 4 |
| | Ap8 | 78 | Ap8-11 | 405-419 | ETQKCFIFNVKPTCL | 48 | 5 |
| | Ap1 | 119 | Ap1-3 | 9-23 | LLSAFEFTYMINFGR | 48 | 6 |
| | Ap1 | 119 | Ap1-9 | 27-41 | QNSDVYRPINEHREH | 48 | 7 |
| | Ap1 | 119 | Ap1-13 | 49-63 | KEYEYPLHQEHTYQQ | 42 | 8 |
| 002 | Ap4 | 208 | Ap4-9 | 189-203 | LMSPMTLDEMRHFYK | 189 | 9 |
| | Ap1 | 181 | Ap1-11 | 37-51 | INEHREHPKEYEYPL | 180 | 10 |
| | Ap4 | 208 | Ap4-10 | 193-207 | MTLDEMRHFYKDNKY | 82 | 11 |
| | Ap1 | 161 | Ap1-12 | 41-55 | REHPKEYEYPLHQEH | 66 | 12 |
| 005 | Ap4 | 310 | Ap4-9 | 189-203 | LMSPMTLDEMRHFYK | 395 | 13 |
| | Ap1 | 325 | Ap1-11 | 37-51 | INEHREHPKEYEYPL | 322 | 14 |
| | Ap8 | 156 | Ap8-11 | 405-419 | ETQKCEIFNVKPTCL | 218 | 15 |
| | Ap8 | 156 | Ap8-12 | 410-419 | CEIFNVKPTCLINNS | 127 | 16 |
| | Ap1 | 325 | Ap1-12 | 41-55 | REHPKEYEYPLHQEH | 118 | 17 |
| | Ap4 | 208 | Ap4-10 | 193-207 | MTLDEMRHFYKDNKY | 118 | 18 |
| | Ap4 | 208 | Ap4-11 | 198-207 | EMRHFYKDNKYVKNL | 65 | 19 |
| | Ap4 | 208 | Ap4-5 | 175-183 | NQYLKDGGFAFPPTE | 50 | 20 |
| 008 | Ap8 | 78 | Ap8-11 | 405-419 | ETQKCEIFNVKPTCL | 48 | 21 |
| 012 | Ap10 | 172 | Ap10-3 | 517-531 | TSNNEVVVKEEYKDE | 233 | 22 |
| | Ap8 | 138 | Ap8-6 | 385-399 | FKADRYKSHGKGYNW | 143 | 23 |

Table 2b illustrates IFN-γ ELISpot activity of AMA1 peptide pools and individual 15-mer peptides within these pools. Shown in the table is the peptide number (pep#) contained in a specific pool that exceeded the ELISpot threshold of 40 SFC/million and were ranked for each volunteer. The activity of the parent peptide pool is shown for comparison. A total of 23 active peptides representing minimal CD8⁺ T-cell epitopes were identified from a total of 91, 15-mer active peptides.

Using NetMHC, putative minimal HLA-A and B-restricted 8-10-mer epitopes within the 23, 15-mers, active in ELISpot that matched the volunteer's HLA super-types, were identified. The identified final 14 peptides are summarized in Table 3. In Table 3, the minimum epitopes are underlined. The 14 predicted minimal epitopes for the corresponding 15-mers were defined as an 8-10 mer. The positive control was commercially obtained CEF-Class I Peptide Pool Plus™ (Anaspec, Freemont, Calif.) and consisted of 32 peptides corresponding to defined HLA class I-restricted T-cell epitopes from cytomegalovirus, Epstein-Barr virus and influenza virus. Negative controls were media with all supplements but no specific stimulant.

The half maximal inhibitory concentration (IC₅₀), in nM, of the predicted epitopes varied according to the matched HLA allele. Some volunteers have HLA alleles in common, for example v002 and v005 are both of the A01 super-type and both recognized the Ap 4 pool peptide 9 (i.e., LMSPMTLDEMRHFY) in ELISpot. The predicted minimum A*0101-restricted epitope in Ap-4-9 was TLDEMRHFY, with an IC₅₀ of 17 (Table 3). Similarly, v001 and v012 share the B44 super-type and both volunteers recognized Ap 10-13 (TSNNEVVVKEEYKDE) in ELISpot that contains a predicted B44-restricted minimum epitope NEVVVKEEY that had a similar IC₅₀ of 15 with both volunteers (Table 3).

After removal of duplicates in the original 23 15-mers, the final number of predicted minimal Class I-restricted epitopes was 14. These are summarized in Table 3. When the predicted binding affinities in IC₅₀'s were examined, 9/14 (64%) of the epitopes were strong binders (<500 nM cut off), consistent with the other NetMHC predictions. Of these 14 minimal epitopes, 2 were predicted to be restricted by HLA-A*0101 and 1 by HLA-A-3002 (A01 super-type); 2 by HLA-A*0201 and 2 by HLA-A*6802 (A02 super-type); 2 by HLA-B*0801 (B08 super-type); 4 by HLA-B*1801 and 1 by HLA-B*4402 (B44 super-type).

TABLE 3
Predicted CD8+-restricted epitopes within AMA1 15 mer
peptides specific for each volunteer

| | Peptide | | | IC₅₀ | | | |
|---|---|---|---|---|---|---|---|
| Volunteer | # | 15 mer | Sequence | nM | HLA | Supertype | Ep # |
| 001 | 1 | TSNNEVVVKEEYKDE | 520-528 | 15 | B*1801 | B44 | 1 |
| | 2 | EDIPHVNEFPAIDLF | 327-335 | 7 | B*1801 | B44 | 2 |
| | 3 | HVNEFPAIDLFECNK | | 7 | | | |
| | 4 | CNKLVFELSASDQPK | 339-346 | 5708 | A*0201 | A02 | 3 |
| | 5 | ETQKCEIFNVKPTCL | 406-414 | 919 | A*0201 | A02 | 4 |
| | 6 | LLSAFEFTYMINFGR | 13-21 | 5 | B*1801 | B44 | 5 |
| | 7 | QNSDVYRPINEHREH | 34-44 | 14991 | B*4402 | B44 | 6 |
| | 8 | KEYEYPLHQEHTYQQ | 51-59 | 39 | B*1801 | B44 | 7 |
| 002 | 9 | LMSPMTLDEMRHFYK | 194-202 | 17 | A*0101 | A01 | 8 |
| | 10 | INEHREHPKEYEYPL | 47-55 | 18 | B*0801 | B08 | 9 |
| | 11 | MTLDEMRHFYKDNKY | | | | | |
| | 12 | REHPKEYEYPLHQEH | | | | | |
| 005 | 13 | LMSPMTLDEMRHFYK | | | | | |
| | 14 | INEHREHPKEYEYPL | | | | | |
| | 15 | ETQKCEIFNVKPTCL | 405-415 | 142 | A*6802 | A02 | 10 |
| | 16 | CEIFNVKPTCLINNS | 410-419 | 617 | A*6802 | A02 | 11 |
| | 17 | REHPKEYEYPL | | | | | |
| | 18 | MTLDEMRHFYKDNKY | | | | | |
| | 19 | EMRHFYKDNKYVKNL | 198-207 | 4742 | A*0101 | A01 | 12 |
| | 20 | NQYLKDGGFAFPPTE | 175-183 | 142 | B*0801 | B08 | 13 |
| 008 | 21 | ETQKEIFNVKPTCL | | | | | |
| 012 | 22 | TSNNEVVVKEEYKDE | | | | | |
| | 23 | FKADRYKSHGKGYNW | 389-397 | 201 | A*3002 | A01 | 14 |

In Table 3, the 15-mer peptides that were recognized by the volunteers in Table 2, were analyzed by NetMHC to predict potential high affinity binding minimal CD8⁺ T cell epitopes within each 15-mer. Each minimal epitope was specific for known HLA-alleles within each super-type. Since the HLA typing analysis only identified super-types, not individual alleles, we have assumed that each volunteer's HLA was specific for the predicted alleles. Those minimal epitopes with the strongest binding affinities for the HLA super-type of each volunteer were selected. The Table shows the minimal epitopes color-coded according to known HLA alleles and their assignment to the HLA super-types of each volunteer. After removing redundant overlapping sequences, 14 HLA-specific epitopes were predicted, belonging to 4 super-types A01, A02, B08 and B44. In order to ascertain the phenotype of responding T-cell populations in Table 2b, CD4⁺ and CD8⁺ depletion studies were conducted. The results of this study are shown in Table 4.

PBMC population depletion was conducted using anti-human CD4+ and anti-CD8⁺ coated Dynabeads™ M-450 (Dynal, Great Neck, N.Y.) following the manufacturer's instructions. Mock depletion was done with Dynabeads coated with Sheep anti-mouse IgG. Flow cytometry confirmed that T-cell subset depletions were >99% in all experiments. The data is presented as the % effect (decrease) in activity after T cell subset depletion.

As shown in Table 4, CD8+ T cell depletion resulted in a 56-100% reduction in ELISpot IFN-γ activity with the 15-mers. Therefore, the results confirm that at least one minimal CD8⁺-restricted epitope for each volunteer was contained within the 15-mers that were recognized in the context of at least one of the super-types A1, A2, B44 and B8. The results with the same peptide in different volunteers generally confirmed our results in the initial screen shown in Table 2b that the same epitope would be active in volunteers who share common HLA alleles. For example, Ap-1-11 was recognized by v002 and v005 who share B8 super-types. Similarly, Ap-10-13 was recognized by v001 and v012 who share B44. The effect of depletion of CD4⁺ T-cells was much lower (0-57%). In v001, CD4+ depletion led to an increase in ELISpot activity that may be the result of removal of suppressor mechanisms.

TABLE 4
ELISpot IFN-γ activity of AMA1 15 mer peptides is largely mediated by CD8+
T cells (minimum epitope is bold and underlined)

| | Pool- | | | Control | | |
|---|---|---|---|---|---|---|
| Volunteer | Peptide | 15 mer | Supertype | dep | CD4 dep | CD8 dep |
| V001 | A1 | | | 155 | 222(+43%) | 53(−65%) |
| | A1-3 | A1-LLSAFEFTYMINFGR | B44 | 85 | 58(−32%) | 37(−56%) |
| | A1-13 | A1-KEYEYPLHQEHTYQQ | B44 | 72 | 168(+133%) | 17(−76%) |
| | A10-13 | A10-TSNNEVVVKEEYKDE | B44 | 103 | 252(+148%) | 13(−88%) |
| V002 | A1 | | | 187 | 148(−21%) | 5(−97%) |
| | A1-11 | A1-INEHREHPKEYEYPL | B08 | 185 | 152(−18%) | 11(−94%) |
| V005 | A1 | | | 267 | 173(−35%) | 11(−96%) |
| | A1-11 | A1-INEHREHPKEYEYPL | B08 | 233 | 145(−37%) | 0(−100%) |
| | A4 | | | 273 | 117(−57%) | 0(−100%) |
| | A4-9 | A4-LMSPMTLDEMRHFYK | A01 | 204 | 137(−32%) | 1(−100%) |
| V012 | A10 | | | 48 | 37(−23%) | 0(−100%) |
| | A10-13 | A10-TSNNEVVVKEEYKDE | B44 | 79 | 90(+14%) | 0(−100%) |

The predicted minimal epitopes were synthesized, and tested in ELISpot assays to confirm that they were functionally active. Each epitope was compared to its parent peptide pool to determine whether the epitopes represented some or all of their ELISpot activity. This is illustrated in Table 5. Since PBMC were limited, PBMC were sourced from other bleeds. To validate this approach, we first compared the peptide-specific ELISpot activities of the different bleeds. PBMC's at 4 months, 7 months and 10 months generally had comparable activities to PBMC at 1 month (Table 5). Since activities of 1 month PBMC from v005 with Ap4-11 and Ap-4-5 (65, 50 sfc/m) were low, these cells were not used. PBMC from the remaining 4 immunized volunteers were stimulated with 12/14 HLA super-type matched predicted minimal peptides (epitopes 1-11 and 14) as well as with the original parent peptide pool.

TABLE 5
ELISpot IFN-γ activity of the original peptide pool
and the derived 8-10 mer epitopes

| Vol | PeptidePool | ELISpot 1m | ELISpot 4m | Ep # | Predicted epitope | ELISpot 4m |
|---|---|---|---|---|---|---|
| 001 | Ap10 | 193 | 155 | 1 | NEVVVKEEY | 344 |
| | Ap7 | 131 | 200 | 2 | NEFPAIDLF | 224 |
| | Ap7 | 131 | 200 | 3 | KLVFELSA | 169 |
| | Ap8 | 78 | 104 | 4 | TQKCEIFNV | 172 |
| | Ap1 | 119 | 133 | 5 | FEFTYMINF | 121 |
| | Ap1 | 119 | 133 | 6 | SDVYRPINEH | 114 |
| | Ap1 | 119 | 222 | 7 | YEYPLHQEH | 222 |
| | | ELISpot 1m | ELISpot 4m | | | ELISpot 4m |
| 002 | Ap4 | 208 | 114 | 8 | TLDEMRHFY | 51 |
| | Ap1 | 161 | 96 | 9 | HPKEYEYPL | 69 |
| | | ELISpot 1m | ELISpot 10m | | | ELISpot 10m |
| 008 | Ap8 | 78 | 57 | 10 | ETQKCEIFNV | 21 |
| | Ap8 | 78 | 57 | 11 | EIFNVKPTCL | 37 |
| | | ELISpot 1m | ELISpot 7m | | | ELISpot 7m |
| 012 | Ap8 | 138 | 108 | 14 | RYKSHGKGY | 11 |
| | Ap1 | 53 | 6 | 7 | YEYPLHQEH | 113 |
| | Ap1 | 53 | 6 | 5 | FEFTYMINF | 4 |
| | Ap7 | 72 | 80 | 2 | NEFPAIDLF | 49 |
| | Ap10 | 172 | 151 | 1 | NEVVVKEEY | 104 |

The ELISpot activities of the peptide pools with cells taken 1 month after immunization were generally similar to responses using other bleeds. Two exceptions were noted with v012 using cells taken at 7 months. Volunteer v012 recognized Ap-1 using frozen cells at 1 month after immunization but not at 7 month after immunization. However, v012 did recognize one of the two B44-restricted epitopes within Ap-1, epitope 7 (i.e., YEYPLHQEH), but not epitope 5 (i.e., FEFTYMINF). Also, v012 recognized Ap-8 at 7 months but not epitope 14 RYKSHGKGY within this pool. We interpret these results as suggesting that the kinetics of the immune response varies over time as measured by peptide pools or individual minimal epitopes.

As summarized in Table 5, PBMCs from volunteers v001 and v002 recognized minimum epitopes 1-9, while v008 was negative with epitopes 10 and 11, and v012 was negative with epitope 14 by our criteria. Taken together, using all four volunteers at different time points, 9/14 of the minimal epitopes were recognized by PBMCs from one or more volunteers. Six of the 9 confirmed epitopes active in ELISpot (Table 5) had predicted binding affinities less than 500 nM (Table 3), and 3 were >500 nm, again agreeing with NetMHC predictions. Based on the depletion studies (Table 4) we suggest that these 9 epitopes are recognized by CD8+ T cells.

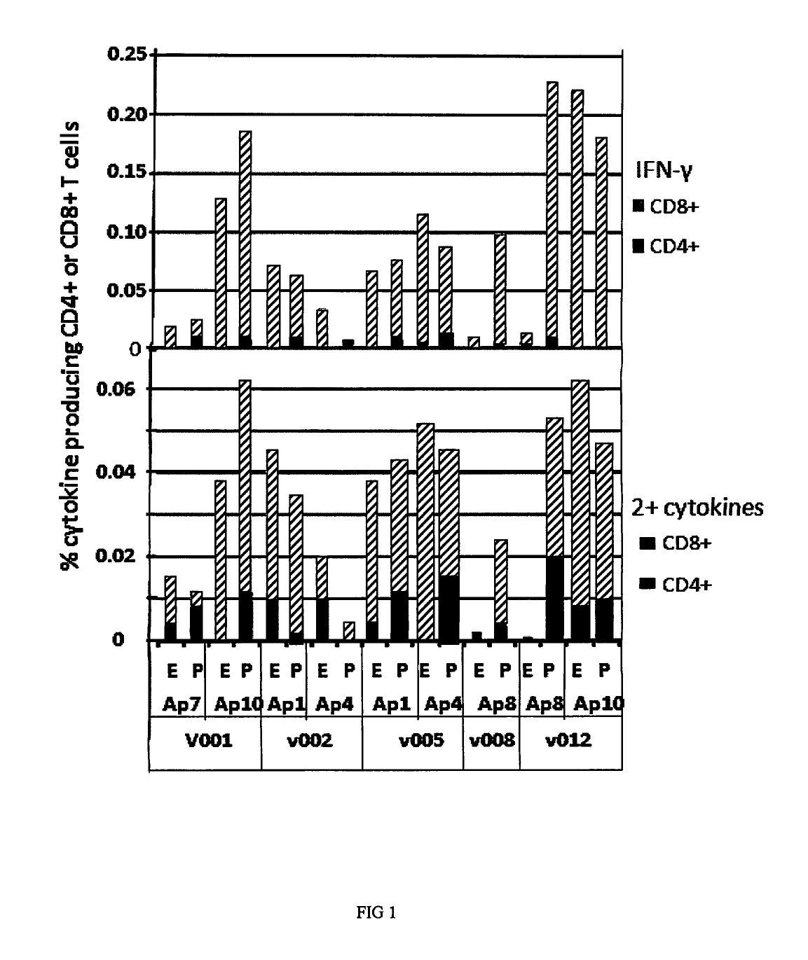

To confirm that these minimal epitopes were recognized by CD8⁺ T cells, we used intracellular cytokine staining (ICS) assays to measure peptide-specific CD4+ and CD8⁺ T cell IFN-γ responses. The results of these studies are summarized in (FIG. 1).

ICS was performed on PBMCs with costimulatory antibodies anti-CD28 and anti-CD4⁺9d (obtained from BD Bioscience™, San Jose, Calif.). The peptides were used at the same concentrations as in ELISpot assays. Stimulants were added t cells and incubated at 37° C. with 5% CO₂ for 2 hr. Golgi Plug™ (Brefeldin A) (BD Bioscience™, San Jose, Calif.) was added at a final concentration of 0.6 μL/mL and incubated at 37° C. with 5% CO₂ overnight. Cells were stained with anti-CD3 (Alexa Fluor 700™), anti-CD4 (PerCP™), and anti-CD8 Pacific Blue™ (obtained from BD Bioscience™, San Jose, Calif.). and 1 μg/mL of live/dead fixable blue dye, incubated and washed. Cells were permeabilized with Cytofix/Cytoperm™ solution (obtained from BD Bioscience™, San Jose, Calif.), incubated and washed. Cells were stained intracellularly with anti-CD3 (Alexa Fluor 700™), anti-CD4 (PerCP™), and anti-CD8 Pacific Blue™, anti-IFN-γ FITC, anti-TNF PE, and anti-IL2 APC, incubated and washed. Cells were resuspended and the entire available sample was acquired by flowcytometry. The gating strategy included progressively measuring total cells; viable cells only; lymphocytes; T cells; CD4⁺ CD8⁺ populations; and a specific cell type expressing a specific cytokine. Histograms were used to determine the total production of IFN-γ, IL2, and TNF for the CD4⁺ and CD8⁺ populations. Boolean gates were used to determine cells producing combinations of cytokines.

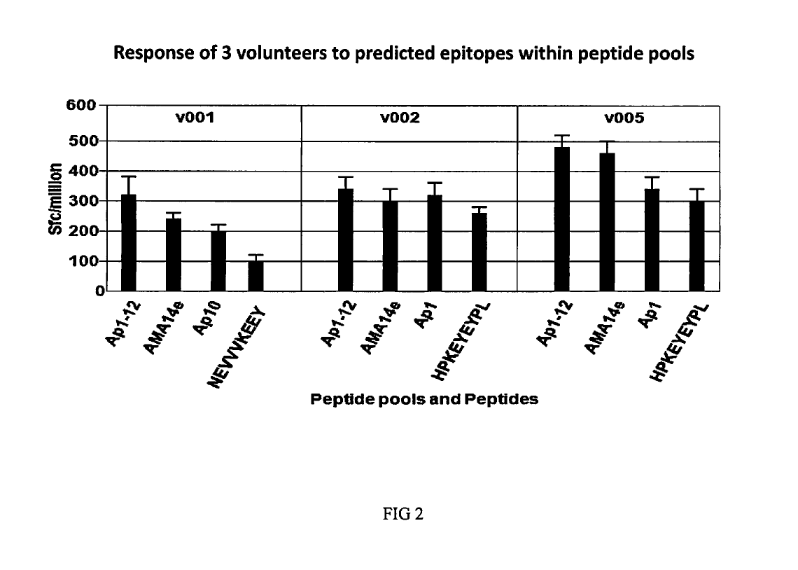

In these studies, T cells containing IFN-γ, IL2, or TNFα, were gated into different subsets: IFN-γ+, IL2+, TNFα−; IFN-γ+, IL2−, TNFα+; IFN-γ+, IL2+, TNFα+; or IFN-γ+, IL2−, TNFα− T cells, and stimulated with the predicted epitopes and their parent peptide pool. The frequency of CD8⁺ T cells producing total IFN-γ was far greater than CD4⁺ T cell responses (FIG. 1, upper panel), which were extremely low. Four of the tested minimal peptide epitopes induced equal or more CD8⁺ IFN-γ than the parent peptide pool: Ap7 and epitope NEFPAIDLF (v001, but very low responses); Ap10 and NEVVVKEEY (v001 and v012); Ap1 and HPKEYEYPL (v002 and v005); and Ap4 and TLDEMRHIFY (v002 and v005) (FIG. 2, upper panel). However, the Ap8 epitopes ETQKCEIFNY and RYKSHGKGY (see FIG. 2b) induced very weak CD8⁺ IFN-γ responses with v008 and v012 (FIG. 1, upper panel), although the parent peptide pool Ap8 induced strong CD8⁺ IFN-γ responses; similar outcomes were noted by ELISpot (Table 4).

The pattern of multifunctional CD8⁺ T cell responses with each minimal epitope (FIG. 1, lower panel) were generally similar to total CD8⁺ IFN-γ responses (FIG. 1, upper panel), although they were often lower. Again, Ap8 epitopes ETQKCEIFNY and RYKSHGKGY (FIG. 2b) did not appear to induce multifunctional responses with v008 and v012.

Having shown that volunteers generally responded to the predicted minimal epitopes, we wished to demonstrate the feasibility of using pooled minimal epitopes to evaluate CD8⁺ T-cell AMA1 responses in immunized volunteers. First we wanted to establish that each volunteer could recognize its matched immunogenic epitope in the presence of other non-matching epitopes; that is, non-binding epitopes do not interfere with specific HLA-restricted binding.

To test this we used the three volunteers who consistently gave high responses to peptide pools and used four different stimulants for ELISpot assays. (1) Ap1-12: all 153 15-mer epitopes spanning the entire length of AMA1, tested final concentration at 1.25 mg/ml; (2) AMA14e: all the identified 14 minimal epitopes mixed and tested at final concentration 10 μg/mL; and (3) selected 15-mer peptide pools that were strongly recognized by the tested volunteers: (v001, Ap10; v002 Ap1; and v005 Ap1). Also test was (4), the minimal epitope within those selected 15-mer peptide pools previously shown to have ELISpot activity. As shown in FIG. 2, AMA14e was as active or nearly as active in inducing ELISpot responses with v001, v002 and v005 as a mixture of all 153 15-mers (Ap1-12), and particularly in v001 and v005 higher than ELISpot responses to the minimal epitope

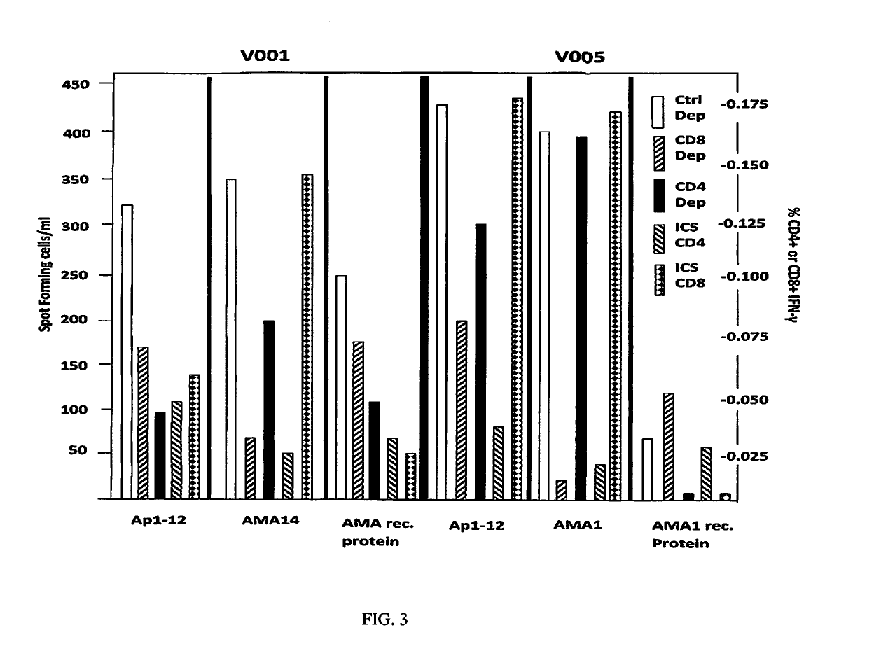

Finally, we wanted to confirm that indeed the responses to AMA14e were mediated by CD8⁺ T cells. We compared activity to AMA14e in ELISpot depletion assays and ICS CD4⁺ or CD8⁺ T cell IFN-γ assays with Ap1-12 and recombinant AMA1 protein with v001 and v005 (FIG. 3). CD8⁺ T cell depletion reduced ELISpot activity to AMA14e by about 85% in v001 and 95% in v005. Similarly AMA14e much more strongly induced CD8⁺ than CD4⁺ T cell IFN-γ in ICS. In contrast, Ap1-12 induced both CD4⁺ and CD8⁺ T cell responses, whereas AMA1 recombinant protein only induced CD4⁺ T cell responses (FIG. 3). This suggests that all of the peptides in AMA14e could operate together, in an immunogenic formulation, to induce a CD8⁺ T cell response. Additionally, these data suggest that the AMA14e might be a suitable reagent to demonstrate CD8⁺ T cell immunogenicity of AMA1 based vaccines in future vaccine trials where volunteers have HLA alleles that would recognize the minimal epitopes in AMA14e.

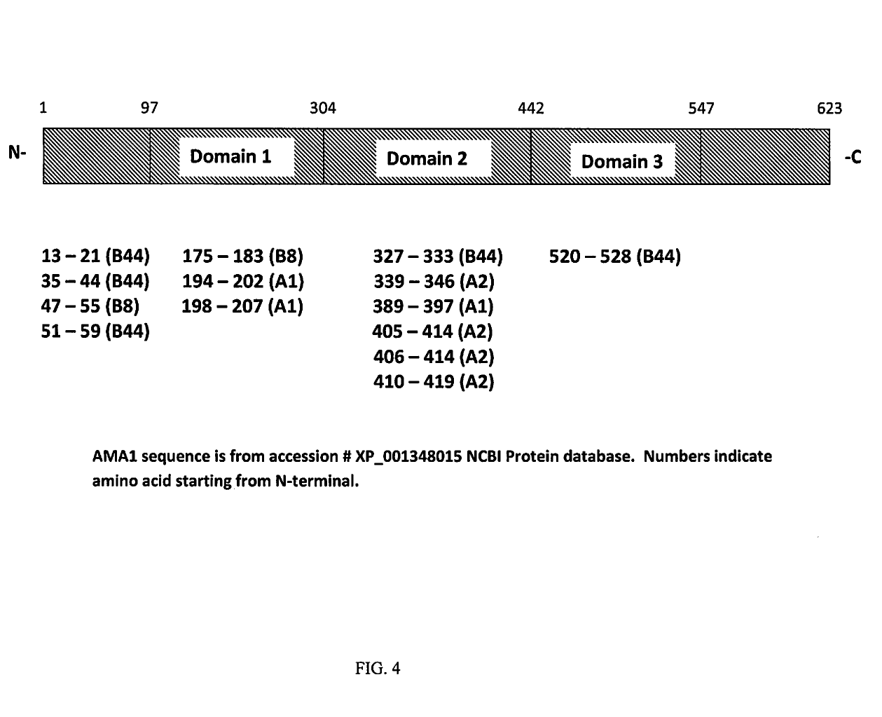

The positions of the predicted minimal epitopes are shown in FIG. 4. Five of the predicted minimal epitopes are localized in the signal sequence and pro-domain that is cleaved off first during cell invasion, whereas the remaining 11 epitopes are localized within Domains I, 2 and 3 that are next cleaved during invasion. The apparent clustering of epitopes may be due to incomplete characterization of the total T epitopes in AMA1. Three of the 14 predicted CD8⁺ minimal epitopes were contained with proliferative epitopes identified in Kenya: epitope 5 FEFTYMINF and PL186; peptide 8 TLDEMRHFY and PL189; peptide 14 RYKSHGKGY and PL193. The other 11 predicted epitopes were largely or wholly independent of these proliferative epitopes.

### Example 2

### Identification of Regions of AMA1 Capable of Inducing High CD8⁺ T Cell Response

The regions of AMA-1 encompassing the 12 pools, discussed in Example 1, are further illustrated in FIG. 5, associating the pools with the specific AMA1 amino acid sequence. Additionally, 14 predicted minimal epitopes from the 15-mer were defined as 8-10 mers. Table 5, above, illustrates the individual peptides and the predicted minimal 8-10 mer epitopes.

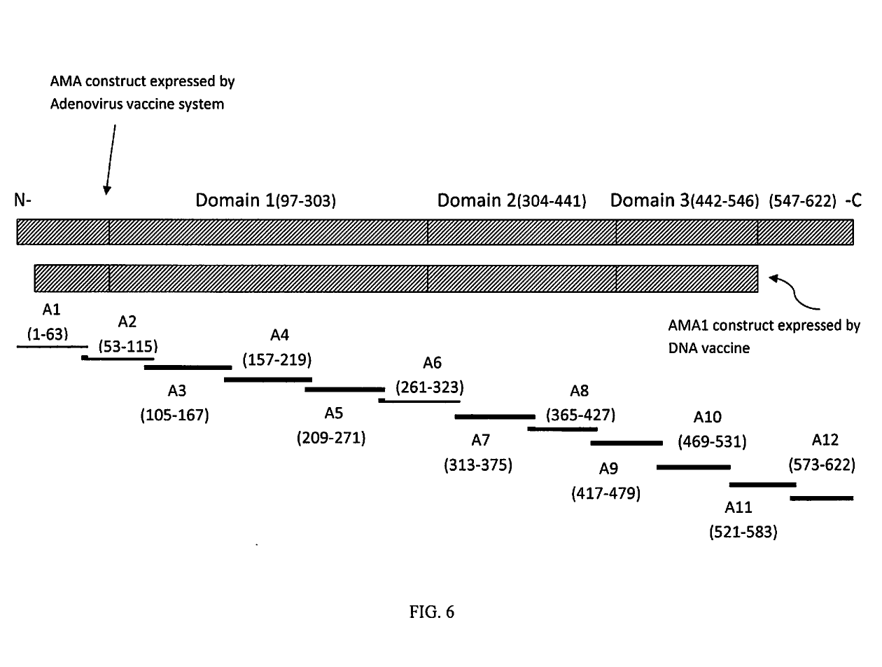

In a preferred embodiment, from the twelve AMA-1 peptide pools covering the entire span of AMA1, two pools were identified, predicated on PBMC responses from immunized volunteers that elicited the greatest response in ELISPOT assays. FIG. 6 illustrates the regions of AMA-1 protein represented by each of the peptide pools Ap1-12.

To more clearly define immunological active regions of AMA1, PBMCs were isolated from AMA1 immunized volunteers. Two groups of volunteers, i.e., Gp 1 (from example 1, above) and Gp2, received 2×10¹⁰ particle units of AdPfCA (NMRC-M3V-Ad-PfCA, NMRC+Multi-antigen Multi-stage, Malaria Vaccine+Adenovector+P. falciparum CSP & AMA1 antigens). The vaccine is a combination of two separate recombinant Ad5 constructs, one expressing full length CSP and the other expressing full length apical membrane antigen 1 (AMA1). AdPfCA expressed the AMA1 regions A1 through A12. Twenty-eight (28) days later, PBMC's were collected.

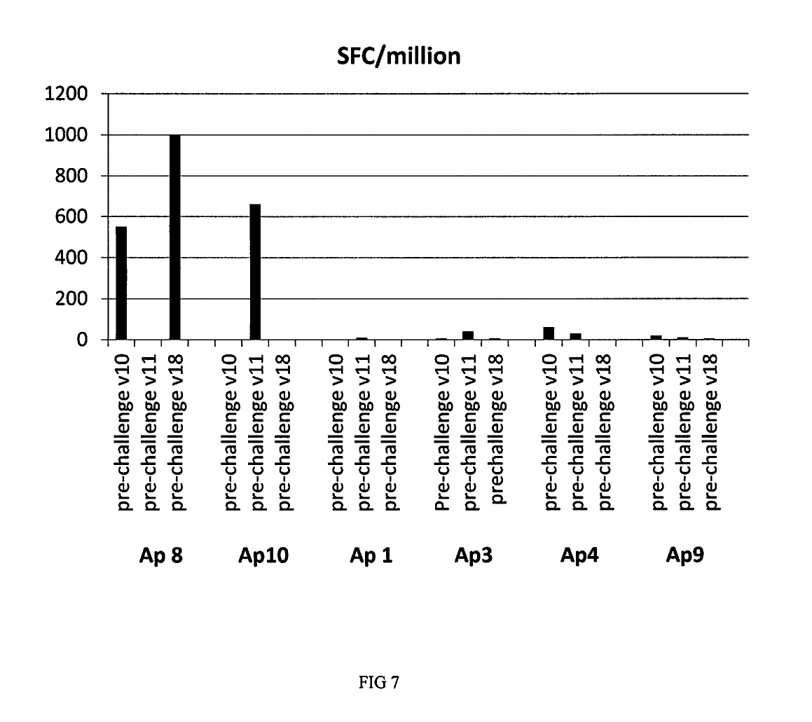

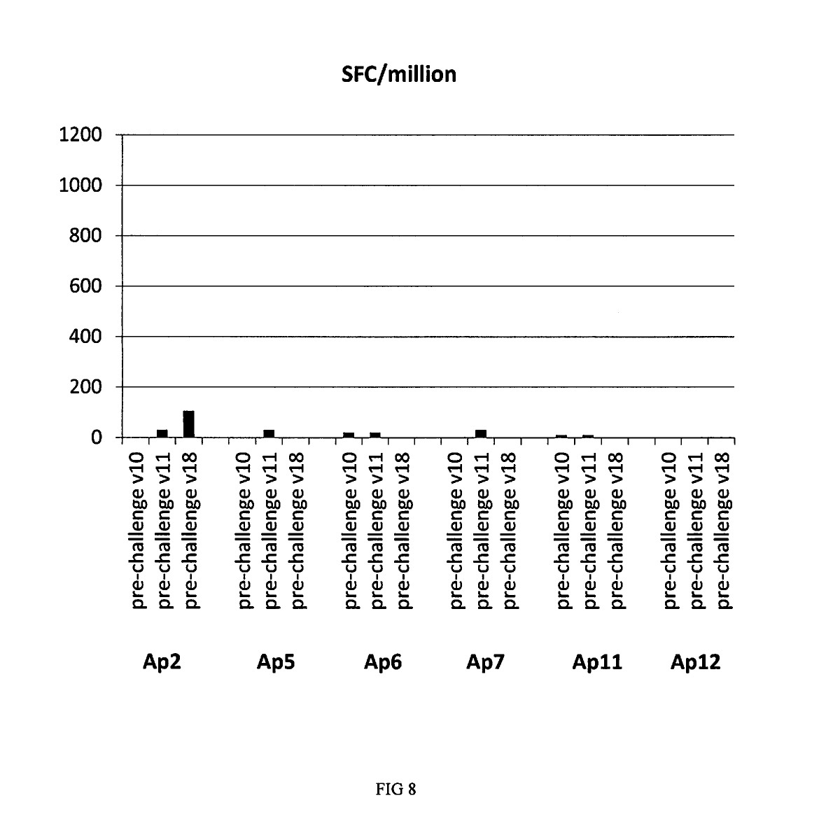

In a third group (termed preCh in FIG. 7 and FIG. 8), fifteen volunteers were primed with three doses (2 mg/dose) with one month between doses. Sixteen weeks after the final DNA priming dose, each volunteer received 2×10¹⁰ particle units of AdPfCA. PBMC's were collected on day 22-23 after receiving AdPfCA. The region of the AMA gene expressed by the DNA expression plasmid is also illustrated in FIG. 6. The DNA vaccine expressed approximately half of the A1 region of AMA1, the entire regions of A2 through A10 and a portion of A11. As in Gp 1 and Gp 2, AdPfCA expressed the AMA1 regions A1 through A12.

The stimulation of circulating antigen-specific T cell lymphocytes was evaluated by measurement of γ-IFN induction by ELISPOT. The ELISpot assay was conducted using thawed cryopreserved PBMC at 200K cells per well. Pre-challenge cells were taken at day 21 and day 22 post AdPfCA vaccination. Cells were suspended in 100 ml complete medium and were stimulated with AMA1 peptides suspended in 100 ml of complete medium. Peptide concentrations used in assays was 10 μg/ml of each peptide tested. Cultures were incubated for 36 h at 37° C., 5% CO2. Depending on availability of cells, each PBMC sample was assayed in duplicate, triplicate, or quadruplicate and the number of IFN-γ-secreting cells recognized as spot-forming cells (SFC) was enumerated using an automated ELISpot reader. In duplicate assays, all values were used in analysis. For triplicate or quadruplicate assays, outliers were rejected if any single value contributed more than 50% of the standard deviation of the triplicate (or quadruplicate) and if its value was three-fold greater or less than the average of the remaining two (or three) values. The mean spot forming cells (SFCs) obtained in negative control wells were subtracted from the value of each test well from the same sample. Negative counts generated by this background subtraction were converted to zero. The mean spot number of the test sample was then calculated and expressed as SFC/million PBMCs. Values obtained with pre-vaccinated samples were subtracted from the post-vaccination values.

FIGS. 7 and 8 show the results of exposing PBMCs from volunteers exposed to AdPfCA to pools of AMA1 peptides. FIG. 7 shows the response, in SFC/million of high responding pools. As shown in FIG. 7, of the pools, Ap8 and Ap10 elicited the highest response. FIG. 8 shows the response of relatively lower responding pools.

As illustrated in FIG. 7, PBMCs from three volunteers, all of which were in the third group (i.e., termed “pre-challenge”), i.e., volunteer #10, 11 and 18, responded vigorously to two of the PfAMA1 pools, Ap 8 and Ap10. As indicated above, these individuals had been primed with three doses of a DNA vaccine containing AMA1 and then boosted with AdPfCA. This observation suggests that domain 2 and domain 3 (see FIG. 4) may be particularly important antigenic regions of the AMA1 molecule.

Therefore, a preferred embodiment of the invention is an immunogenic composition comprising one or more of the polypeptide regions of AMA1 contained within domain 2 and domain 3, defined by SEQ ID Nos 16 and 17, respectively. Additionally, another embodiment includes an immunogenic composition comprising one or more of the polypeptides defining the specific regions of AMA1 encompassed with within Ap 8 and Ap10, SEQ ID No. 1 and 2, respectively.

In a further study, volunteers were immunized with AdCA. PBMCs were collected either 22/23 days after immunization with AdCA or 4 or 12 weeks after challenge with P. falciparum. In this study, T-cell activity was based on the SFC/million of IFN-γ producing cells. The results of these studies are summarized in Table 7. In Table 7, the HLA binding motif indicated is underlined.

The results of this study, summarized in Table 6, identified other CD8⁺ epitopes, associated specific HLA class I genes. Of particular interest is that depletion of CD8⁺ T-cells from either of Ap8 or Ap10 pool populations resulted in 89% to 94%, respectively. This is in contrast to that observed for the entire Ap1-12 region, where CD8⁺ T-cell depletion resulted in only a 49% to 66% reduction. These results clearly indicate the extent of induction of CD8⁺ T-cells by Ap8 and Ap10 eliciting. Peptides Ap8-6 and Ap8-7, both containing HLA class I gene A1 recognition motifs, induced mostly CD 8⁺ T-cell response. In fact, depletion of this T-cell population resulted in a 99-100% reduction in response. Similarly, the minimal epitope contained in Ap10, NSTCRFFVCK, also stimulated a significant T-cell response, which was primarily attributable to a CD8⁺ T-cell response. Table 7 summarizes the association between sequence identification numbers and epitopes or peptide pool regions.

TABLE 6

| | | | | | CD8⁺ Depletion | CD4⁺ depletion |
|---|---|---|---|---|---|---|
| Vol. ID | | | | Unfract. | % change | % change |
| (HLA | HLA | | | PBMC | (SFC/10⁶: | (SFC: |
| genotype) | binding | Bleed | peptide | (SFC/10⁶) | test/control) | test/control) |
| 1550-10 | | 12 wk | Ap1-Ap12 | | 66% (52/153) | 82% (27/153) |
| | | post | | | | |
| | | challenge | | | | |
| | | | Ap8 | | 89% (12/112) | 39% (68/112) |
| | A1 | 12 wk post | Ap8-6 | | 100% | 26% (81/109) |
| | (B*5701) | challenge | FKADRYKSHGKGYNW | | (0/109) | |
| | A1 | 12 wk Post | Ap8-7 | | 99% | 26% (54/72) |
| | (B*5701) | challenge | RYKSHGKGYNWGNYN | | (1/72) | |
| | A1 | 12 wk post | Ap8 | 131 | | |
| | (B*5701) | challenge | | | | |
| | A1 | 12 wk post | YKSHGKGYNW | 144 | | |
| | (B*5701) | challenge | | | | |
| | A1 | 12 wk post | KSHGKGYNW | 159 | | |
| | (B*5701) | challenge | | | | |
| 1550-11 | | Pre-challenge | Ap1-12 | | 49% (83/161) | 59% (67/161) |
| (A11, A68/ | | | | | | |
| B50, B55) | | | | | | |
| | | Pre-challenge | Ap10 | | 99% (4/414) | 33% (278/414) |
| | | 4 wk post | Ap1-12 | | 70% (37/126) | 78% (28/126) |
| | | challenge | | | | |
| | | 4 wk post | Ap10 | | 100% (0/207) | 34% (91/138) |
| | | challenge | | | | |
| | A1 | 4 wk post | NSTCRFFVCK | | 100% (0/207) | 26% (153/207) |
| | (A*1101) | challenge | | | | |
| 1550-18 | | Pre-challenge | Ap-1-12 | | 94% (19/320) | 2% (326/320) |
| A2/B44, | | | | | | |
| B58 | | | | | | |
| | | Pre-challenge | Ap8 | | 100% (1/383) | +27% (486/383) |
| | | 12 wk post | Ap8 | 158 | | |
| | | challenge | | | | |
| | B58 | 12 wk post | YKSHGKGYNW | 271 | | |
| | (B*5801) | challenge | | | | |
| | B58 | 12 wk post | KSHGKGYNW | 181 | | |
| | (B*5801) | challenge | | | | |

TABLE 7

| Peptide or | | | | | |
|---|---|---|---|---|---|
| epitope | Sequence | Epitope | Peptide or epitope | Sequence | Epitope |
| sequence | ID number | # | sequence | ID number | # |
| Pool Ap8 | 1 | | SDVYRPINEH | 11 | 6 |
| Pool Ap 10 | 2 | | YEYPLHQEYH | 12 | 7 |
| ETQKCEIFNV | 3 | 10 | TLDEMRHFY | 13 | 8 |
| EIFNVKPTCL | 4 | 11 | HPKEYEYPL | 14 | 9 |
| RYKSHGKGY | 5 | 14 | MRHFYKDNK | 15 | 12 |
| NEVVVKEEY | 6 | 1 | YLKDGGFAF | 16 | 13 |
| NEFPAIDLF | 7 | 2 | NSTCRFFVCK | 17 | Ap10 |
| | | | | | epitope |
| KLVFELSA | 8 | 3 | YKSHGKGYN | 18 | Ap8 |
| | | | | | epitope A |
| TQKCEIFNV | 9 | 4 | KSHGKGYNW | 19 | Ap8 |
| | | | | | epitope B |
| FEFTYMINF | 10 | 5 | AMA1 domain 2 | 20 | |
| | | | AMA1 domain 3 | 21 | |

Table 3 shows the predicted CDC T-cell epitopes contained in specific peptide pools Ap 1-12 and their associated HLA super-family restriction. Because of the demonstrated importance of Ap8 and Ap10 in the immune response to AMA1, an embodiment of the invention is the incorporation of one or more of the minimal epitopes encompassed within domain 2 and 3 and, more specifically, within Ap 8 or Ap10. As an illustrative example, one or more of the polypeptides with amino acid sequences represented by SEQ ID No. 3, 4, 5, 16, and 17, contained in Ap 8 or SEQ ID No. 6 and 18, contained in Ap 10, can be utilized in an immunogenic formulation. Alternatively, DNA encoding these peptides can be inserted into a DNA plasmid or viral expression system, which can serve as a component of an immunogenic composition against malaria.

### Example 3

### Use of Epitopes in Vaccine Candidate Evaluation and as Components in Immunogenic Formulations

Class I restricted T-cells from PBMC's from immune volunteers responded vigorously to AMA1 pools, specifically from Ap-8 and Ap-10. From this result, these regions are of particular importance in inducing an immune response. As such, a preferred embodiment is an immunogenic composition, capable of inducing an immune response in mammals, comprising one or more polypeptides encompassing all or an immunogenic portion of the regions contained in domain 2 (SEQ ID No. 20); domain 3 (SEQ ID No. 21); Ap8 (SEQ ID No. 1) and Ap10 (SEQ ID No. 2). A further embodiment of the invention is the incorporation of one or more of the epitopes represented by SEQ ID Nos. 3-19 into immunogenic formulations against malaria.

An additional embodiment is to enable anti-malaria immunity to as large a demographic population as possible. To this end, this embodiment includes the incorporation of epitopes that further contain specific HLA class I binding motifs encompassing significant portions of population groups. In the current invention, the identified epitopes are restricted by 4 HLA supertypes that, together, are expressed on 100% of Caucasians and at least 27% of African Americans.

We previously demonstrated that a single dose of Ad-PfCA, an adenovirus serotype 5-vectored malaria vaccine, induced stronger CD8⁺ than CD4⁺ T cell IFN-γ responses, though weak antibody responses, to its component antigens CSP and AMA1. Therefore, as an additional embodiment, one or more of the polypeptides represented by SEQ ID Nos 1-21 can be encoded by DNA and incorporated into one or more DNA plasmid expression systems or viral expression systems and expressed from a nucleic acid based immunogenic formulation against malaria. For example, one or more of the polypeptides can be expressed from an adenovirus vector to induce an immunogenic response against malaria in mammals, such as in humans. Alternatively, DNA encoding one or more of the epitopes, represented by SEQ ID Nos. 3-19 can be inserted into DNA plasmid or viral expression systems as a component of an immunogenic formulation. In this embodiment, specific, CD8⁺ T cell responses would be elicited from individuals of defined HLA population distribution. It is advantageous to develop peptides that are recognized in conjunction with as many important HLA super-families as possible in or order to afford protection to as large a population as possible. Therefore, it is contemplated, in another preferred embodiment, that the inventive polypeptides could be utilized with other HLA-restricted polypeptides.

A further embodiment of the invention is a method of inducing an immune response utilizing an immunogenic composition containing one or more the peptides of SEQ ID No. 1 through 21. The method comprises administering the immunogenic composition, with or without adjuvant, either as a subunit vaccine or by expressing the peptides as a component of a DNA or viral expression system.

In a preferred embodiment, the contemplated method includes administration of one or more priming immunizations or one or more boosting immunizations of a composition comprising one or more polypeptides with amino acid sequences selected from SEQ ID Nos. 1-21. In another embodiment, the composition comprises one or more isolated nucleic acid molecules inserted into suitable expression vectors. The nucleic acid molecules in this embodiment encode one or more of the polypeptides with amino acid sequences of SEQ ID Nos. 1-21. The embodiment also contemplates that one or more priming or one or more boosting immunizations could comprise administration of irradiated sporozoites.

It is contemplated that suitable expression vectors would be selected from the group consisting of DNA plasmid, alphavirus replicon, adenovirus, poxvirus, adeno-associated virus, cytomegalovirus, canine distemper virus, yellow fever virus and retrovirus. In another embodiment, the priming immunization vector is an alphavirus and the boosting immunization is a non-alphavirus vector. The non-alphavirus vector can be poxvirus, adenovirus, adeno-associated virus and retrovirus. The poxvirus can be cowpox, canarypox, vaccinia, modified vacinia Ankara, or fowlpox. Alternatively, the priming immunization can be comprised of an expression vector that is a DNA plasmid or an adenovirus with the boosting immunization selected from the group consisting of adenovirus, adenovirus that is heterologous to the priming adenovirus, poxvirus and one or more polypeptides, wherein the polypeptides have amino acid sequences selected from SEQ ID No. 1-21. Furthermore, the alphavirus replicon can be a preparation selected from the group consisting of RNA replicon, DNA replicon and alphavirus replicon particles. The alphavirus can be Venezuelan Equine Encephalitis Virus, Semliki Forest virus and Sindbis Virus.

### Example 4

### Use of Specific Regions of AMA1 as a Screen for Important T-Cell Epitopes

The structure of P. falciparum AMA1 has been demonstrated (Bai, et al., Proc. Natl. Acad. Sci. USA, 102: 12736-41 (2005); Pizarro, et al., Science, 308: 408-11 (2005)) and is relatively conserved among Plasmodium species in terms of its three domains. However, there is strain variation within Plasmodium falciparum (Polly, et al., Genetics, 165: 555-61 (2003); Escalante, et al., Mol. Biochem. Parasitol., 125: 83-90 (2002); Cortes, et al., Infect. Immun., 71: 1416-1426 (2003)), reflected by strain-specificity of inhibitory antibodies (Polley, et al., Vaccine, 23: 718-728 (2004); Kennedy, et al., Infect. Immun., 70: 6948-6960 (2002); Remarque, et al., Trends Parasitol., 24: 74-84 (2008)). The variability of many of the proliferative epitopes (Lal, et al., Infect. Immun., 64: 1054-1059 (1996); Udhayakumar, et al., Am. J. Trop. Med. Hyg., 65: 100-107 (2001)) is consistent with antigenic variation under immune pressure (Remarque, et al., Infect. Immun., 76: 2660-2670 (2008)).

Based on the sequence, structure and variation of AMA1 (Remarque, et al., Trends Parasitol., 24: 74-84 (2008)), CD8⁺ highly variable epitopes within AMA1, including Domain 2, and Domain 3, may be of value in evaluating vaccine protective immunity. Immune pressure might induce variability (Takala, S. L. and C. V. Plowe, Parasite Immunol., 31: 560-573 (2009)). Therefore, it is possible that those CD8⁺ T epitopes with the most variability may be under the most immune pressure. For example the polymorphism of other malaria antigens, such as CSP, has been well documented. It has been suggested that this phenomenon is important for development of a successful vaccine (Takala, S. L. and C. V. Plowe, Parasite Immunol., 31: 560-573 (2009); Thera, et al., PLoS ONE, 3: e1465 (2008)). Therefore, antigenic regions within domain 2 and 3, including specific T-cell epitopes, may be of particular value in evaluating vaccine compositions for their protective value, as well as HLA restriction.

In the absence of immunological correlates of protection, the most promising focus is to develop vaccines that give more powerful CMI and antibody responses, as well as ensuring that these responses are directed to known epitopes in the vaccine antigens. Such assays, particularly involving CD8⁺ T cell responses the produce IFN-γ, and multifunctional responses (more than 2 cytokines per cell), have been associated with protection in other diseases including HIV (Valor, et al., Vaccine, 26: 2738-2745 (2008); Barisal, et al., J. Virol, 82: 6458-6469 (2008); Karanam, et al., Vaccine, 27: 1040-1049 (2009); Perales, et al., Mol. Ther., 16: 2022-2029 (2008); Precopio, et al., J. Exp. Med., 204: 1405-1416 (2007)).

A specific embodiment, therefore, is a method to screen potential vaccines candidates for their efficaciousness against malaria. The method comprises:
1. exposing human lymphocytes to one or more AMA1 polypeptides selected from the group consisting of SEQ ID No. 3-19; and
2. determining CD4⁺ and CD8⁺ T-cell response.
3. recording epitopes responded to and HLA restriction of the epitope.

Determination of responder T-cell populations can be conducted in any number methods. In a preferred embodiment, induction of γ-IFN or other T-cell cytokines are measured by ELISpot assay.

1-16. (canceled)

17. A method of evaluating immunity to malaria vaccine candidate comprising:
a. exposing human lymphocytes to specific Plasmodium falciparum epitopes restricted to specific HLA class I super-families and epitopes from specific HLA class I alleles;
b. determining CD4+ and CD8+ T-cell response to said epitopes;
c. recording epitopes responded to and HLA restriction of the epitope.

18. The method of claim 17, wherein said determination of T-cell response is by ELISpot assay.

19. The method of claim 17, wherein said determination of T-cell response is by
determination of T-cell induction of by cytokine induction.

20. The method of claim 19, wherein said cytokine is IFN-γ.

21. The method of claim 17, wherein said HLA class I super-families are selected from the group consisting of A01, A02, B08, and B44.

22. The method of claim 17, wherein said epitopes restricted to specific HLA class I super-families and epitopes from specific HLA class I alleles comprise one or more polypeptides selected from the group consisting of SEQ ID No. 3, 4, 5, 6, 7, 8, 9, 10, 11, 12, 13, 14, 15, 16, 17, 18 and 19.


SEQUENCE LISTING (22 sequences)

SEQ ID NO: 1 - protein, 63 aa, Plasmodium falciparum
EGFKNKNASMIKSAFLPTGAFKADRYKSHGKGYNWGNYNTETQKCEIFNVKPTCLINNSSYIA

SEQ ID NO: 2 - protein, 61 aa, Plasmodium falciparum
GNKKIIAPRIFFISDDKDSLKCPCDPEMVSNSTCRFFVCKCVERRAEVTSNNEVVVKEEYK

SEQ ID NO: 3 - protein, 10 aa, Plasmodium falciparum
ETQKCEIFNV

SEQ ID NO: 4 - protein, 10 aa, Plasmodium falciparum
EIFNVKPTCL

SEQ ID NO: 5 - protein, 9 aa, Plasmodium falciparum
RYKSHGKGY

SEQ ID NO: 6 - protein, 9 aa, Plasmodium falciparum
NEVVVKEEY

SEQ ID NO: 7 - protein, 9 aa, Plasmodium falciparum
NEFPAIDLF

SEQ ID NO: 8 - protein, 8 aa, Plasmodium falciparum
KLVFELSA

SEQ ID NO: 9 - protein, 9 aa, Plasmodium falciparum
TQKCEIFNV

SEQ ID NO: 10 - protein, 9 aa, Plasmodium falciparum
FEFTYMINF

SEQ ID NO: 11 - protein, 10 aa, Plasmodium falciparum
SDVYRPINEH

SEQ ID NO: 12 - protein, 9 aa, Plasmodium falciparum
YEYPLHQEH

SEQ ID NO: 13 - protein, 9 aa, Plasmodium falciparum
TLDEMRHFY

SEQ ID NO: 14 - protein, 9 aa, Plasmodium falciparum
HPKEYEYPL

SEQ ID NO: 15 - protein, 9 aa, Plasmodium falciparum
MRHFYKKNK

SEQ ID NO: 16 - protein, 9 aa, Plasmodium falciparum
YLKDGGFAF

SEQ ID NO: 17 - protein, 10 aa, Plasmodium falciparum
NSTCRFFVCK

SEQ ID NO: 18 - protein, 9 aa, Plasmodium falciparum
YKSHGKGYN

SEQ ID NO: 19 - protein, 9 aa, Plasmodium falciparum
KSHGKGYNW

SEQ ID NO: 20 - protein, 137 aa, Plasmodium falciparum
RKNLQNKFGLWVDGNCEDIPHVNEFPAIDLFECNKLVFELSASDQPKQYEQHLTDYEKIKEGFKNKNASMIKSAFLPTGAFKADRYKSHGKGYNWGNTETQKCEIFNVKPTCLINNSSYIATTASHPIEVENNFPCS

SEQ ID NO: 21 - protein, 100 aa, Plasmodium falciparum
LYKDEIMKEIERESKRIKLNDNDDEGNKKIIAPRIFFISDDKDSLKCPCDPEMVSNSTCRFFVCKCVERRAEVTSNNEVVVKEEYKDEYADIPEHKPTYD

SEQ ID NO: 22 - protein, 617 aa, Plasmodium falciparum
MRKLYCVLLLSAFEFTYMINFGRGCNYWEHPYQNSDVYRPINEHREHPKEYEYPLHQEHTYQQEDSGEDENTLQHAYPIDHEGAEPAPQEQNLFSSIEIVERSNYMGNPWTEYMAKYDIEEVHGSGIRVDLGEDAEVAGTQYRLPSGKCPVFGKGIIIENSNTTFLTPVATGNQYLKDGGFAFPPTEPLMSPMTLDEMRHFYKDNKYVKNLDELTLCSRHAGNMIPDNDKNSNYKYPAVYDDKDKKCHILYAAQENNGPRYCNKDESKRNSMFCFRPAKDISFQNYTYLSKNVVDNWEKVCPRKNLQNKFGLWVDGNCEDIPHVNEFPAIDLFECNKLVFELSASDQPKQYEQHLTDYEKIKEGFKNKNASMIKSAFLPTGAFKADRYKSHGKGYNWGNTETQKCEIFNVKPTCLINNSSYIATTASHPIEVENNFPCSLYKDEIMKEIERESKRIKLNDNDDEGNKKIIAPRIFFISDDKDSLKCPCDPEMVSNSTCRFFVCKCVERRAEVTSNNEVVVKEEYKDEYADIPEHKPTYDKMKIIIASSAAVAVATILMVYLYKRKGNAEKYDKMDEPQDYGKSNSRNDEMLDPEASFWGEEKRASHTTPVLMEKPYY